(19) United States
(12) Patent Application Publication
(10) Pub. No.: US 2019/0330588 A1
(43) Pub. Date: Oct. 31, 2019

(54) Increased Efficiency and Diversity of Microbes Cultured from Environmental Samples

(71) Applicant: NOVOZYMES BIOAG A/S, Bagsvaerd (DK)

(72) Inventors: Yaowei Kang, Chapel Hill, NC (US); Jessica Smith, Holly Springs, NC (US)

(73) Assignee: NOVOZYMES BIOAG A/S, Bagsvaerd (DK)

(21) Appl. No.: 16/089,150

(22) PCT Filed: Mar. 2, 2017
(86) PCT No.: PCT/US2017/020333

§ 371 Date: Sep. 27, 2018

Related U.S. Application Data
(60) Provisional application No. 62/316,201, filed on Mar. 31, 2016.

(51) Int. Cl. C12N 1/20; C12Q 1/689; C12Q 1/24

(52) U.S. Cl. CPC C12N 1/20; C12N 2500/30; C12Q 1/24; C12Q 1/689

(57) ABSTRACT

Methods for isolating microbes from environmental samples using growth medium containing humic acid and related substances are described. In one example, the isolated microbes have not been cultured previously or were not previously known. The microbes isolated using humic acid can subsequently be cultured on media that does not contain humic acid.

### BACKGROUND

Microbes in some environments are said to be “unculturable.” These microbes cannot be cultured in the laboratory using current techniques. But, DNA sequences obtained from samples from these environments confirm that the microbes are present. In one example, one gram of soil is frequently stated to contain millions to billions of microbes. However, it is also often stated that only 1% of these microbes can be cultured in the laboratory. Because microbes from environmental samples may have a variety of uses, methodologies that could increase the proportion of microbes from environmental samples that could be cultured would be useful.

### SUMMARY

We have found that by adding humic acid or related substances to microbial growth media, that microbes previously thought to be unculturable, as well as previously unknown microbes, can be cultured from environmental samples. Disclosed herein, therefore, are methods for isolating microbes, including unculturable and unknown microbes, from environmental samples using media that contains humic acid, salts thereof, analogs thereof, or peat. In one example, the isolated microbes may be bacteria or archaea. The environmental samples may come from a variety of sources, including soil.

We have also found that, after initial culturing of the microbes using media containing humic acid or related substances, the microbes can be grown on media that does not contain humic acid or related substances.

### BRIEF DESCRIPTION OF THE DRAWINGS

In the accompanying drawings, which are incorporated in and constitute a part of the specification, embodiments of methods and reagents related to methods of isolating microbes from environmental samples using humic acid and related substances are illustrated which, together with the detailed description given below, serve to describe the examples. It will be appreciated that the embodiments illustrated in the drawings are shown for the purpose of illustration and not for limitation. It will be appreciated that changes, modifications and deviations from the embodiments illustrated in the drawings may be made without departing from the spirit and scope of the invention, as disclosed below.

FIG. 1 illustrates an example experiment showing increased efficiency of bacterial colony formation on agar-containing medium after plating serial dilutions of soil samples on R2A medium (left) or R2A medium containing humic acid (right). The two plates at the top of the figure were plated with the same serial dilution from the soil sample. The two plates at the bottom were plated with the same dilution from the soil sample, but a different dilution than the plates shown at the top of the figure.

FIG. 2 illustrates an example experiment showing that bacteria isolated on media containing humic acid can subsequently be grown on media that does not contain humic acid. The two plates shown in panel A of the figure contain bacterial colonies originally isolated on R2A plates, then transferred to the R2A plates shown in the figure using sterile toothpicks. The four plates shown in panel B of the figure contain bacterial colonies originally isolated on R2A plates that contained 0.5% humic acid, then transferred to the R2A plates (not containing humic acid) shown in the figure using sterile toothpicks.

### DETAILED DESCRIPTION

### Definitions

The following includes definitions of selected terms that may be used throughout the disclosure and in the claims. The definitions include various examples and/or forms of components that fall within the scope of a term and that may be used for implementation. The examples are not intended to be limiting. Both singular and plural forms of terms fall within the definitions.

As used herein, “ambient atmosphere” means the general atmospheric composition in the surrounding area. For example, atmospheric compositions containing 5% CO₂, or less than 10% O₂ are not considered ambient herein. Atmospheric compositions of less than 1% CO₂, or about 21% O₂ are considered ambient atmospheric conditions herein.

As used herein, “archaea” means prokaryotic organisms that do not have peptidoglycan in their cell walls, and have lipids in their membranes that do not contain fatty acids.

As used herein, “agar” means a gelatinous substance, generally derived from seaweed, and used in culture media to provide media that is solid or semisolid in consistency. In one example, agar concentrations of about 0.5-1.5% (weight/volume) in media may be used for microbial culture plates. Herein, agar is considered a type of gelling agent.

As used herein, “about” means±10% with respect to the stated value or parameter.

As used herein, an “analog,” of a first substance (e.g., humic acid) refers to a second substance that is structurally similar to the first substance, but with some differences. An analog may be synthetic.

As used herein, “bacteria” means prokaryotic organisms that have peptidoglycan in their cell walls, and have lipids in their membranes that contain fatty acids.

As used herein, “colony” means a visible cluster of microbes, generally on the surface of a solid or semisolid medium (e.g., medium containing agar), and probably originating from division of a single cell. A colony formed by bacteria may be called a “bacterial colony.”

As used herein, “culturing,” when referring to microbes, means to grow or proliferate the microbes. “Cultured from” refers to the source from which the growing microbes were obtained. “Cultured in” or “cultured on” refers to where the microbes are cultured. For example, a microbe that is cultured on a medium containing agar, is generally being grown on a medium that is solid or semisolid in consistency.

As used herein, “determine” means to establish or find out. “Determining” is an act to establish or find out. Something that has been established or found out may be said to be “determined.”

As used herein, “dilution,” when used as a noun, refers to a liquid that contains a reduced concentration of a thing as compared to the liquid when undiluted.

As used herein, “diversity” means variety or different. For example, a first microbe population may be said to be more diverse or to have more diversity than a second microbe population. In one example, this may mean that the first population contains more different species of microbes or more different genera of microbes than the second population.

As used herein, “efficiency,” when referring to culturing of microbes, means a ratio of colonies formed per number of microbes plated (e.g., on a medium that contains agar) that is higher in one condition (e.g., in presence of humic acid) than in another condition (e.g., without humic acid).

As used herein, “environmental sample” means a sample taken or acquired from any part of the environment (e.g., habitat). Example environmental samples may be from soil, water, wood, insects, worms, activated sludge, and the like.

As used herein, “enrichment” means to increase the number or proportion of a thing in a sample.

As used herein, “exclude” means to prohibit or leave out.

As used herein, “facilitate” means to help something to occur or to make something easier.

As used herein, “gelling agent” refers to substances that are added to liquid to cause the liquid to become solid or semisolid in consistency. A variety of these substances exist. Example gelling agents may include agar, agarose, alginic acid, carrageenan, gelatin, gellan gum, guar gum, xanthan gum, and the like.

As used herein, “humic acid” refers to a principal component of humic substances (fulvic acid and humin are other principal components of humic substances) that is soluble in dilute alkali but which becomes insoluble as the pH becomes acidic. Substances “related to” humic acid may include humic acid analogs, synthetic humic acids, and may also include peat.

As used herein, “indicate” means to point out or to show.

As used herein, “isolate” means to separate or segregate from. “Isolating” is an act to separate or segregate from. In one example, a single microbe may be isolated from a soil sample that contains many different microbes.

As used herein, “medium,” with reference to a culture medium for a microbe, refers to compositions for supporting growth of microbes. Example growth medium may include liquid media (e.g., broths) or solid/semisolid media (e.g., agar-containing media).

As used herein, “microbe” means cells that are not mammalian (e.g., bacteria, fungi, yeast, archaea).

As used herein, “obtain” means to get or acquire.

As used herein, “peat” generally refers to partially decomposed vegetable/plant matter.

As used herein, “petri dish” means a shallow, generally transparent dish with a flat lid, used for culture of microbes.

As used herein, “plating” refers to applying an environmental sample, microbes from an environmental sample, or dilution of the environmental sample or microbes, to solid or semisolid microbial culture medium (e.g., agar-containing medium). “Plated” refers to something that has been applied to solid or semisolid microbial culture medium.

As used herein, “portion” means a part of a whole.

As used herein, “prior” means before.

As used herein, “prokaryote” means single-celled organisms that do not have a membrane-bound nucleus.

As used herein, “recognizing” means knowing about.

As used herein, “salt” refers to an ionic form of a substance.

As used herein, “selecting” means choosing.

As used herein, “soil” generally refers to a mixture of organic matter, minerals, gases, liquids, microbes, and the like, present in the upper layer of the earth.

As used herein, “subsequent” refers to occurrence of something in time, after the occurrence of something else.

As used herein, “synthetic” refers to something that is synthesized, rather than naturally occurring. A synthetic substance may be an analog.

As used herein, “taxonomic group” refers to hierarchical groups into which related organisms are classified. For example, a specific genus is a taxonomic group, as is a specific species. There are a variety of different genera and species.

As used herein, “transfer” means to move from one place to another.

As used herein, “unculturable,” when referring to a microbe, means unable to be cultured, using current technologies (i.e., technologies prior to this disclosure). A microbe that is considered unculturable may eventually be cultured, for example, when technologies are improved. In one example, a microbe cultured using the methods disclosed herein may not have been cultured previously. In the context of this disclosure, such a microbe would be called unculturable because it was the technological improvement disclosed herein that resulted in the microbe being cultured.

As used herein, “unknown,” when referring to a microbe, means that the microbe was not previously known to exist. Herein, a microbe isolated using humic acid may be called unknown because it was not known prior to disclosure of the methods disclosed herein. An unknown microbe may form a new genus or species, for example. Unknown microbes have not previously been reported to exist. Known microbes, on the other hand, are known to exist and may be part of known taxonomic groups.

As used herein, “use” means to employ or put into service. “Using” is an act to employ or put into service. Something that has been employed or put into service may be said to be “used.”

### Environmental Samples and Unculturable Microbes

Microbes (e.g., archaea, bacteria, fungi, yeast) likely exist in all parts of the biosphere as well as on and in many living things (i.e., microbiota). Samples from the biosphere/microbiota may be procured and the microbes therein may be detected in the samples without culturing. Disclosed herein, however, are methods for increasing the efficiency and/or the diversity of microbes that are cultured from these samples.

Typical environmental samples may be obtained from the earth (e.g., soil, permafrost, sediments), water (e.g., fresh water, seawater, deep-sea vents), air, materials in the environment (e.g., decaying materials like rotting wood, compost), from the surface (e.g., skin) of animals (e.g., mammals, insects, worms), from inside (e.g., digestive tract, gut) animals (e.g., humans), from plants or plant-associated material (e.g., plant roots, plant seeds), possibly from outer space, and the like. Environmental samples may also be procured from man-made or artificial environments (e.g., wastewater, activated sludge, hospitals, and ventilation systems). In general, the environmental samples may be procured from natural environments, artificial environments, from attempted replications of natural environments, and the like. In one example, the environmental sample is soil. In one example, the environmental sample is water.

A proportion of the microbes in samples from some of these environments may be “unculturable.” Unculturable microbes are so named because, using current technologies, the microbes cannot be cultured (e.g., in the laboratory). The unculturable microbes may be detected in the samples, however, because techniques like DNA sequencing can detect genomes of the unculturable microbes. There are also microbes in some environmental samples whose DNA has not been detected. These microbes may be called “unknown” because they are not currently known to exist. An unknown microbe may be culturable or may be unculturable. An unculturable microbe may be known or may be unknown. In one example, unculturable and/or unknown microbes may be prokaryotic (e.g., archaea, bacteria). In one example, unculturable and/or unknown microbes may be eukaryotic (e.g., fungi, yeast).

When a conserved nucleotide sequence (e.g., a 16s rRNA nucleotide sequence) has been obtained from a microbe from an environmental sample, for example, it may be possible to determine if the conserved sequence originates from an unculturable and/or unknown microbe. Various publically available nucleotide sequence databases contain, for example, 16S rRNA sequences that are generally designated as originating from either culturable or unculturable microbes. A 16S rRNA sequence, for example, can be used to query these databases for identical sequences (e.g., using BLASTN). Sequence matches resulting from these sequence queries (i.e., retrieved sequences in the databases that have identity with the query sequence) will generally be identified as originating from either culturable or unculturable microbes and may help determine whether the query sequence originates from a microbe known to be culturable or unculturable. Lack of a sequence match between a query sequence and sequences in a database may indicate that the 16S rRNA query sequence originates from a microbe that has not previously been described (i.e., an unknown microbe).

A “sequence match” between a query sequence and a retrieved sequence may be determined based on identity between the query sequence and the retrieved sequence, the identity generally measurable over a given length (e.g., 99% of the length) of the query sequence. Sequence identity matches may include identity levels between the query and retrieved sequences, for example, of at least 90, 91, 92, 93, 94, 95, 96, 97, 98, 99, or 100%. Lack of a sequence identity match may include identity levels of less than any of these stated levels. In one example, a sequence identity match of at least 94, 95, or 96% between a 16S rRNA query sequence and a sequence retrieved from a database may indicate that the microbe from which the query sequence originated should be classified within the same taxonomic genus as the microbe from which the retrieved sequence originated. In one example, a sequence identity match of at least 94, 95, or 96% between a 16S rRNA query sequence and a retrieved sequence, where the retrieved sequence is indicated to be from an unculturable microbe, may indicate that the microbe from which the query sequence originated should also be classified as unculturable, at least until such a microbe is cultured using the methods disclosed herein.

### Humic Acids, Salts, and Analogs

Soil organic matter may be classified as a humic substance or a non-humic substance. Humic substances are composed of altered or transformed components of plants, animals, microbes, and the like (e.g., decomposed organic matter). Non-humic substances include unaltered remains (e.g., not decomposed) of plants, animals, microbes, and the like. Humic substances are generally thought to include a humic acid component, a fulvic acid component, and a humin component. The humic acid component, and substances that may contain all or part of the humic acid component, is disclosed herein as capable of increasing the efficiency of plating of microbes from environmental samples and/or increasing the diversity of microbes isolated from environmental samples.

These three components of humic substances—humic acid, fulvic acid, and humin—are defined, in part, based on their aqueous solubilities at different pH values. The humic acid component, for example, is generally water soluble at alkaline pH, but becomes less soluble under acidic conditions. In one example, humic acid may be defined as the fraction of humic substances that are water insoluble at pH 2, but are increasingly soluble at higher pH values. The fulvic acid component is generally soluble in water at all pH values. The humin component is generally insoluble at all pH values.

Chemically, humic acid is a complex mixture of weak aliphatic and aromatic organic acids, often containing phenolic and carboxylic substituents. Humic acids (HAs) may be called polydisperse because of their variable chemical features. The molecular sizes of humic acids (HAs) may range, in one example, from approximately about 10,000 to about 100,000. Humic acids (HAs) may readily form salts with inorganic trace mineral elements. Both humic acids and salts thereof can be used and may be active in the methods disclosed herein.

Humic substances, and therefore humic acid, may be components of soil (e.g., humus), peat, lignite, coal, lake and stream sediments, seawater, and shale (e.g., Leonardite). Humic acid may be obtained or extracted from certain of these substances (e.g., convenient sources may be humus rich soil, peat moss, compost) using various methods. Humic acid may also be obtained from systems set up to facilitate degradation of organic materials (e.g., plant material) so that humic acid is produced. Humic acid may also be formed by polymerization of substances like polyphenols. Some of these methods are described in, for example, U.S. Pat. No. 5,854,032. Other methods for extracting or producing humic acids may be used. Humic acids can also be purchased commercially (e.g., Sigma-Aldrich No. 53680). The above-mentioned substances—like peat, lignite, coal, sediments, seawater, shale, and the like—are also within the scope of materials that increase plating efficiency and/or diversity of microbes isolated from environmental samples.

Salts of humic acid are within the scope of materials that can increase the efficiency and diversity of microbes isolated from environmental samples. In one example, formation of salts of humic acid depends on the ability of carboxyl and/or hydroxyl groups therein to dissociate their hydrogen ions and bind to positive cations (e.g., metal cations like iron, copper, zinc, calcium, manganese, magnesium, and the like). Salts of humic acid can be purchased commercially (Sigma-Aldrich No. H16752).

Humic acid analogs and synthetic humic acids (a humic acid analog may also be synthetic) also exist and are within the scope of materials that may increase the efficiency and diversity of microbes isolated from environmental samples. In one example, certain quinones, one being anthraquinone-2, 6-disulfonate (AQDS), are considered analogs of humic acid. Synthetic humic acids can be made by methods known in the art (e.g., V. A. Litvin, R. L. Galagan. “Synthesis and Properties of Synthetic Analogs of Natural Humic Acids.” Russian Journal of Applied Chemistry 85, no. 2, 2012).

Humic acid may be fractionated and some of the fractions may be successfully used in the methods disclosed herein. In one example of fractionating, humic acid is added to an aqueous solution of 0.1 M ammonium bicarbonate at a slightly basic pH. Insoluble material is removed from the mixture. The remaining solution is passed through a filter that retains molecules larger than 5,000 molecular weight on the filter, while molecules smaller than 5,000 molecular weight pass through the filter. The material retained on the filter may be shown to possess the activity of increasing the efficiency of plating of microbes from soil samples and/or increasing the diversity of microbes isolated from soil samples. Other methods of fractionating humic acid may be used.

### Microbial Isolation Using Humic Acids

In one example of the methods disclosed herein, microbes are isolated from an environmental sample using a microbial growth medium that contains humic acid or related substances. Generally, the humic acids may be added to any microbial culture media. In various examples, the microbial media may be designed for culturing a variety of different microbes, including bacteria, fungi, yeast, and archaea. In one example, the culture medium may be known to support growth of bacteria. In one example, the growth media may include R2A, TSA, LB, NA, ISP2, Jensen's, and the like. Addition of humic acids to the media generally increases the efficiency of plating of microbes (i.e., the number of microbes that grow) and/or the diversity of microbes that are cultured from environmental samples, as compared to the efficiency of plating and/or diversity using media without humic acid. In one example, microbes known but previously unculturable, or previously unknown microbes, may be cultured on the media containing humic acid or related substances.

Media used for culturing microbes may be liquid, semisolid or solid. Semisolid or solid medium may be made, in one example, by adding a gelling agent to a liquid medium. A common gelling agent is agar. However, a number of other gelling agents exist and may be used. Examples include agarose, alginic acid, carrageenan, gelatin, gellan gum, guar gum, xanthan gum, and others. Generally, microbes plated on a semisolid or solid medium may divide and form colonies after a time when the medium is placed in an environment conducive to growth of microbes (e.g., 2-3 days incubation at 30° C. in an ambient atmosphere). However, these conditions (e.g., days of incubation, temperature, atmosphere) may vary and may be empirically determined.

Different forms of humic acid may require different concentrations within media to produce increased efficiency of plating and increased diversity of microbes isolated from environmental samples, as compared to media that lacks the humic acids. In one example, concentrations of any of the various humic acid forms above 0% (weight/volume) may be used. In one example, humic acid forms may be used at concentrations above 0% and less than about 5% (e.g., 0.25, 0.50, 1.50, 2.00, 2.50%). In one example, humic acid forms may be used at concentrations above 0% and less than about 0.25% (e.g., 0.10, 0.15, 0.20, 0.25%). In one example, a concentration of humic acid used in the medium is not 0.1% or is above 0.1%. In one example, concentrations of humic acid between about 0-5% or 0.05-2.00% may be used. In one example, a concentration of a salt of humic acid below about 0.25% may be used. In one example, a concentration of peat of about 0.5% may be used.

In one example, a medium that contains a form of humic acid is selected or chosen for use in the disclosed methods because a user may recognize that efficiency and/or diversity of microbes cultured from an environmental sample may be increased by using humic acid in the medium. Subsequent to this recognition, the user may use the medium containing humic acid, for example, to isolate an unculturable microbe and/or an unknown microbe from an environmental sample.

In one example, isolating microbes from an environmental sample occurs after directly culturing a portion of the environmental sample on or in a medium containing humic acid and/or its various forms. “Directly culturing,” in this context, means that intermediary procedures or steps may not generally be needed to obtain increased efficiency of plating and/or increased diversity of isolated microbes. Example intermediary steps could include enrichment steps (e.g., enrichment culture that enriches for fast-growing microbes; dilution culture that enriches for prevalent microbes) that enrich for certain microbes in or from an environmental sample, and/or could include preliminary growth of the microbes from an environmental sample before they are cultured using medium containing humic acid. Note that procedures like storage of environmental samples (e.g., in a refrigerated environment) before growth on media containing humic acid, or making serial dilutions from an environmental sample before applying a part of a serial dilution to medium containing humic acid, are generally not considered intermediary steps in this context. Therefore, in one example, all or a portion of an environmental sample, or dilution thereof, is cultured on or in a medium containing humic acid or one of its forms, without use of intermediary procedures or steps. In other examples, intermediary procedures may be used.

Microbes that are cultured or isolated using humic acid, according to methods of this disclosure, may subsequently be cultured, in one example, on medium that does not include humic acid. In one example, the microbes initially isolated using medium containing humic acid may grow similarly to, or as well as, the microbes did grow on the humic acid-containing medium. In one example, the microbes initially isolated using medium containing humic acid may grow more slowly than they did grow on the humic acid-containing medium. In one example, the microbes initially isolated using medium containing humic acid may not grow on medium that does not contain humic acid (e.g., humic acid may be required for further or subsequent growth). In one example, microbes initially isolated on medium containing humic acid may grow on medium that contains less humic acid than used in the original isolation.

In one example, the microbes cultured or isolated using the disclosed methods may be bacteria or may be archaea. These bacteria and archaea may be from a variety of different genera and species, as disclosed herein. In one example, the bacteria cultured using the disclosed methods may be from any of the following genera: Actinotalea, Amycolatopsis, Aquabacterium, Bacillus, Burkholderia, Caenimonas, Dermacoccus, Leifisonia, Lysinibacillus, Marmoricola, Massilia, Methylobacterium, Mucilaginibacter, Nocardia, Nocardioides, Novosphingobium, Paenibacillus, Phycicoccus, Ramlibacter, Rhizobacter, Rugamonas, Sphingomonas, Streptomyces, Terrabacter, Tetrasphaera, Tumebacillus, and Variovorax. However, many genera and species other than those disclosed herein may be cultured using the methods.

In one example, the microbes cultured using the methods disclosed herein may not be from the order Actinomycetales (e.g., microbes from this order may be excluded). In one example, the microbes cultured using the methods disclosed herein may not be from the phyla Acidobacteria and Verrucomicrobia (e.g., microbes from one or both of these phyla may be excluded). In one example, the excluded Acidobacteria may belong to subdivision 1 only. In one example, the excluded Verrucomicrobia may belong to subdivision 4 only.

Attempts may be made to identify (e.g., the genus and species) the microbes isolated using the methods disclosed herein. In one example, identification may be made after obtaining the nucleotide sequence, or partial nucleotide sequence, of 16S rRNA from bacteria isolated using the methods. As described elsewhere herein, these sequences may be used to query various databases for identical or nearly identical sequences (i.e., retrieved sequences). These methods may enable the isolated microbes from which the particular 16S rRNA originated to be assigned to taxonomic groups. Or, these methods may enable the isolated microbes to be determined to be unculturable and/or unknown. In one example, if it is not possible to assign a microbe to a taxonomic group based on sufficient identity of its 16s rRNA sequence to a sequence in a database, the microbe may be determined to be unculturable and/or unknown. These attempts to classify or assign the isolated microbes may use 16S rRNA sequences alone. In some examples, the 16S rRNA sequences may be used in combination with sequences of other genes, of multiple genes, or even sequences of whole genomes.

### EXAMPLES

The following examples are for the purpose of illustrating various embodiments and are not to be construed as limitations.

### Example 1. Preparation of Media Containing Humic Acid

A variety of microbial media were used in these studies. R2A (Dehydrated R2A Agar, No. DF1826-07-3), TSA (Dehydrated Tryptic Soy Agar, No. DF0369-07-8), LB (Dehydrated Luria-Bertani Agar, No. DF0445-17-4), NA (Oxoid™ Nutrient Agar, No. OXCM0003B) and ISP2 (Dehydrated ISP Medium 2, No. DF0770-17-9) were purchased from Fisher Scientific. Jensen's Medium Agar (No. M710) was from HiMedia Laboratories (Mumbai, India).

Humic acid (No. 53680; Sigma-Aldrich, St. Louis, Mo., USA) or humic acid sodium salt (No. H16752; Sigma-Aldrich, St. Louis, Mo., USA) was added to media at the indicated percentages (weight/volume) prior to autoclaving. After autoclaving, for media containing No. 53680 humic acid, the media was swirled while pouring agar plates to ensure humic acid was uniformly distributed throughout the media.

Various peats were also used in these studies, including: DAKOTA™ Peat (DAKOTA Peat & Equipment, Grand Forks, N. Dak., USA), an unspecified peat from Partac Peat Corporation (Great Meadows, N.J., USA), an unspecified peat from McMaster-Carr Supply Company (Elmhurst, Ill., USA), an unspecified peat from Organic Products Company (Groveland, Fla., USA), and an unspecified peat from our laboratory labeled, “CxC Enviro.”

Fulvic acid was obtained from Kelp4Less.com (Idaho Falls, Id., USA).

The peats and fulvic acid were added to media in the same way as the humic acids, described above.

### Example 2. Environmental Samples

Soil samples were obtained from a forested location in Chapel Hill, N.C., USA. The first soil sample (soil sample A) was procured in October 2015. The second soil sample (soil sample B) was procured in November 2015. The samples were obtained from locations within about 100 feet of one another. The samples contained soil from the soil surface to a depth of about 15 cm. Soil sample B was bulk soil. Sample A, in addition to bulk soil, also contained plant root materials. Soil samples were kept at 4° C. in a refrigerator until use.

In addition to the soil samples, a pond water sample, wood sample from a decaying log, and earthworm sample were also used in these studies. These 3 samples were obtained from locations very near the Chapel Hill, N.C. locations where the soil samples were obtained.

### Example 3. Increased Efficiency of Isolating Bacteria from Soil Using Media Containing Humic Acid

Soil (0.5 g of soil sample A) was added to 50 ml of sterile phosphorus buffer (No. NC00716471; Fisher Scientific) and vigorously mixed. Serial dilutions of the mixture were made in phosphorus buffer and aliquots from the dilutions were cultured on R2A plates or on R2A plates containing 0.5% humic acid (Sigma-Aldrich No. 53680). The plates were incubated at 30° C. for 2-3 days in ambient atmosphere and then examined for formation of bacterial colonies.

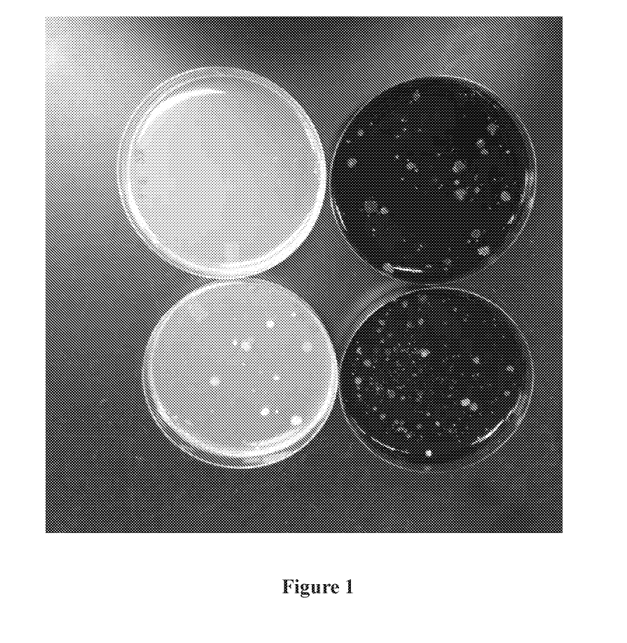

FIG. 1 shows example results from these experiments. In FIG. 1, two R2A plates onto which aliquots from serial dilutions were plated are shown on the left and two R2A+humic acid plates onto which equivalent aliquots were plated are shown on the right (agar containing humic acid is darker in color). The two plates pictured at the top of FIG. 1 were plated with a different dilution than the two plates pictured at the bottom of FIG. 1.

The results show that there were more microbial colonies on the R2A+humic acid plates than were on the R2A plates. This was an indication that addition of humic acid increased the efficiency of culturing bacteria from these soil environmental samples. Generally, in part because R2A media is designed to support bacterial growth, the majority of colonies formed in these studies were colonies of bacteria.

To test whether the increased efficiency of isolating bacteria from the sample was specific to R2A plates, or was a more general phenomenon observed with other types of media, a number of different types of agar medium, with and without 0.5% humic acid added, were used in similar plating experiments. The different types of media used are described in Example 1. As already described, serial dilutions of soil made in phosphate buffer were cultured on the media. After incubation of the plates at 30° C. for 2-3 days, colonies were counted. Colony counts obtained from plates onto which aliquots from different serial dilutions were plated were adjusted based on the dilution factor. Example results from these experiments are shown in Table 1.

TABLE 1
Microbe isolation efficiency on various media
with and without 0.5% humic acid (HA)

| | | Relative number |
|---|---|---|
| | Average number | of colonies (for each |
| | of colonies | medium compared to same |
| Media | per plate¹ | medium without HA)² |
| TSA | 81 | 1.0 |
| TSA + HA | 256 | 3.2 |
| LB | 44 | 1.0 |
| LB + HA | 256 | 6.0 |
| NA | 214 | 1.0 |
| NA + HA | 460 | 2.1 |
| ISP2 | 26 | 1.0 |
| ISP2 + HA | 101 | 3.9 |
| R2A | 309 | 1.0 |
| R2A + HA | 1183 | 3.8 |
| Jensen's | 215 | 1.0 |
| Jensen's + HA | 573 | 2.7 |

¹Means from colony counts from 3 plates.
²Average colony counts on plates containing humic acid were normalized to colony counts on the same medium that did not contain humic acid.

These data show that, for each of the media tested, that addition of 0.5% (weight/volume) humic acid increased the number of colonies on the plates. In these experiments, using 6 different media, the increases in colonies with humic acid as compared to no humic acid ranged from 2.1-fold (NA+HA) to 6.0 fold (LB+HA). These data indicate that the effect of increasing the efficiency of plating these soil microbes with humic acid is not specific to a certain medium but, instead, is a general phenomenon that works with many different types of culture medium.

Another set of experiments were performed to determine the concentrations of humic acid that optimized the efficiency of plating bacteria from the soil sample. Serially-diluted samples from the sample were plated and cultured, as described above, on R2A plates containing a range of humic acid concentrations (Sigma-Aldrich No. 53680).

Tables 2 and 3 represent example data obtained from these studies.

TABLE 2
Microbe plating efficiency on R2A medium containing
different concentrations of humic acid (HA)

| | | Relative number colonies |
|---|---|---|
| Concentration | Average number | (for each humic acid |
| of HA in | of colonies | concentration compared |
| media (%) | per plate¹ | to no humic acid) |
| 0 | 115 | 1.0 |
| 0.05 | 180 | 1.6 |
| 0.5 | 398 | 3.5 |
| 5.0 | 58 | 0.5 |

¹Means from colony counts from 3 plates.

TABLE 3
Microbe plating efficiency on R2A medium containing
different concentrations of humic acid (HA)

| | | Relative number colonies |
|---|---|---|
| Concentration | Average number | (for each humic acid |
| of HA in | of colonies | concentration compared |
| media | per plate¹ | to no humic acid) |
| 0 | 62 | 1.0 |
| 0.25 | 376 | 6.1 |
| 0.5 | 337 | 5.4 |
| 1.0 | 340 | 5.5 |
| 2.0 | 215 | 3.5 |

¹Means from colony counts from 3 plates.

The data from Tables 2 and 3 show that concentrations of humic acid from 0.05% to at least 2% in the R2A plates increased the number of colonies as compared to R2A plates containing no humic acid. Since 0.05% humic acid, the lowest concentration tested, yielded 1.6 times more colonies than plates containing no humic acid (Table 2), it is likely that humic acid concentrations between 0 and 0.05% also would produce an increased efficiency of microbe plating. The highest concentration of humic acid tested that yielded an increased colony number was 2% (Table 3; 3.5 times more colonies than plates with no humic acid). The next highest concentration of humic acid tested was 5% (Table 3), which yielded a decrease in colony number as compared to plates containing no humic acid (0.5 times the number of colonies on plates without humic acid). These data suggest that, at some concentration above 2%, humic acid no longer increases the efficiency of plating. These data suggest that the concentrations of humic acid at which efficiency of plating bacteria from the example soil sample was increased, was somewhere between 0.05-0.5% on the low end, and somewhere between 0.5-1.0% on the high end. In general, we used 0.5% humic acid in many of our studies.

In addition to humic acid, the sodium salt of humic acid (No. H16752; Sigma-Aldrich, St. Louis, Mo., USA) was tested for its ability to increase the efficiency of isolating bacteria from soil. This salt of humic acid, unlike the non-salt form, became soluble after addition to media and during autoclaving the media.

Similar to the studies with the non-salt form of humic acid, these humic acid salt studies were performed by plating and culturing serially-diluted soil, as previously described. Example results from these studies are shown in Tables 4, 5, and 6.

TABLE 4
Microbe plating efficiency on R2A medium containing
humic acid (HA) or humic acid sodium salt (HASS)

| Form and | | Relative number colonies |
|---|---|---|
| concentration of | Average number | (for each humic acid |
| humic acid in | of colonies | concentration as compared |
| R2A medium | per plate¹ | to no humic acid) |
| R2A | 53 | 1.0 |
| R2A + 0.5% HA | 260 | 4.9 |
| R2A + 0.5% HASS | 11 | 0.2 |

¹Means from colony counts from 3 plates.

TABLE 5
Microbe plating efficiency on R2A medium containing
humic acid (HA) or humic acid sodium salt (HASS)

| Form and | | Relative number colonies |
|---|---|---|
| concentration of | Average number | (for each humic acid |
| humic acid in | of colonies | concentration as compared |
| R2A medium | per plate¹ | to no humic acid) |
| R2A | 38 | 1.0 |
| R2A + 0.25% HA | 97 | 2.6 |
| R2A + 0.25% HASS | 36 | 1.0 |

¹Means from colony counts from 3 plates.

TABLE 6
Microbe plating efficiency on R2A medium containing
humic acid (HA) or humic acid sodium salt (HASS)

| Form and | | Relative number colonies |
|---|---|---|
| concentration of | Average number | (for each humic acid |
| humic acid in | of colonies | concentration as compared |
| R2A medium | per plate¹ | to no humic acid) |
| R2A | 52 | 1.0 |
| R2A + 0.250% HA | 115 | 2.2 |
| R2A + 0.050% HASS | 57 | 1.1 |
| R2A + 0.010% HASS | 74 | 1.4 |
| R2A + 0.002% HASS | 70 | 1.3 |

¹Means from colony counts from 3 plates.

Our finding was that a 0.5% concentration of the sodium salt form of humic acid in R2A medium decreased the number of colonies as compared to no humic acid (Table 4), even though this concentration of the non-sodium salt form of humic acid increased the number of colonies. At concentrations below 0.25% (e.g., 0.05%, 0.01%, and 0.002%), the sodium salt form of humic acid did increase the efficiency of microbe colony formation (Table 6). However, at least in these example studies, this increase was not as high as were the increases with optimal concentrations of the non-salt form of humic acid.

### Example 4. Effects of Peat and Fulvic Acid on Efficiency of Isolating Bacteria from Soil

In addition to humic acid, other substances were tested for their ability to increase the colony formation efficiency of microbes from an example environmental sample. Peat from various sources was tested, as was fulvic acid (peats and fulvic acid are described in Example 1). These substances were incorporated into R2A plates at 0.5% (weight/volume) as described in Example 1. Serial dilutions of soil sample A in phosphorus buffer were plated and cultured on the media, as described in Example 3. The example results obtained with the peats and fulvic acid were compared to those obtained with humic acid (Sigma-Aldrich No. 53680). Example results from these studies are shown in Table 7.

TABLE 7
Microbe plating efficiency on R2A medium with and without
0.5% (weight/volume) various peats or fulvic acid

| | | Relative number |
|---|---|---|
| | Average number | of colonies (for each |
| | of colonies | medium compared to |
| Media | per plate¹ | R2A)² |
| R2A | 309 | 1.0 |
| R2A + humic acid | 1183 | 3.8 |
| R2A + DAKOTA ™ peat | 326 | 1.1 |
| R2A + CxC Enviro peat | 540 | 1.7 |
| R2A + Partac peat | 450 | 1.5 |
| R2A + Organic Products peat | 149 | 0.5 |
| R2A + fulvic acid | 246 | 0.8 |
| R2A + McMaster-Carr peat | 58 | 0.2 |

¹Means from colony counts from 3 plates.
²Average colony counts on plates were normalized to colony counts on R2A medium.

The data show that, while fulvic acid did not appear to increase colony formation as compared to media not containing fulvic acid, that some of the peats tested did increase the efficiency of colony formation.

### Example 5. Efficiency of Isolating Microbes from Environmental Samples Other than Soil Using Humic Acid

Experiments were performed to test the effect of humic acid on isolation of microbes from environmental samples other than soil. Samples from pond water, decaying wood, and an earthworm were procured, as described in Example 2.

Serial dilutions of the pond water sample were made as were done for soil as described in Example 3. For the decaying wood sample, 2 g of the wood sample were added to 100 ml of phosphorus buffer, vigorously mixed, and serial dilutions were made. For the earthworm sample, the earthworm was rinsed in ethanol, 3× rinsed in water, then stored in 40% glycerol at −80° C. The earthworm was then thawed, crushed using a mortar and pestle until homogeneous, and serial dilutions were made.

For all of these samples, aliquots from the serial dilutions were cultured on R2A plates and on R2A plates containing 0.5% humic acid (Sigma-Aldrich No. 53680), in triplicate. The plates were incubated at 30° C. for 3 days in ambient atmosphere and colonies were counted. Example results from the pond water sample are shown below in Table 8.

TABLE 8
Microbe plating efficiency from pond water on R2A medium
with and without 0.5% (weight/volume) humic acid (HA)

| | Number of | Number of | Relative number |
|---|---|---|---|
| Experiment | colonies on | colonies on | colonies R2A + HA |
| number | R2A¹ | R2A + 0.5% HA¹ | to R2A² |
| 1 | 70 | 84 | 1.2 |
| 2 | 147 | 180 | 1.2 |

¹Means from colony counts from 3 plates.
²Average colony counts on plates containing humic acid were normalized to colony counts on the same medium that did not contain humic acid.

The data show that there was about a 20% increase in colonies on R2A plus humic acid compared to R2A for the pond water sample. We did not see an increase in colony number in presence of humic acid for the decaying wood and earthworm samples (the number of colonies on R2A was the same as on R2A+HA).

### Example 6. No Requirement of Microbes Isolated in Presence of Humic Acid for Continued Presence of Humic Acid

A study was performed to determine whether microbes isolated from an environmental sample (e.g., soil) using media containing humic acid, required continued presence of humic acid in order to grow. To perform these experiments, randomly-selected single colonies, isolated after plating soil on either R2A plates or on R2A plates containing 0.5% humic acid (Sigma-Aldrich No. 53680), as described in Example 3, were transferred onto R2A plates that did not contain humic acid, using sterile toothpicks. Growth of the microbes transferred to the R2A plates was scored.

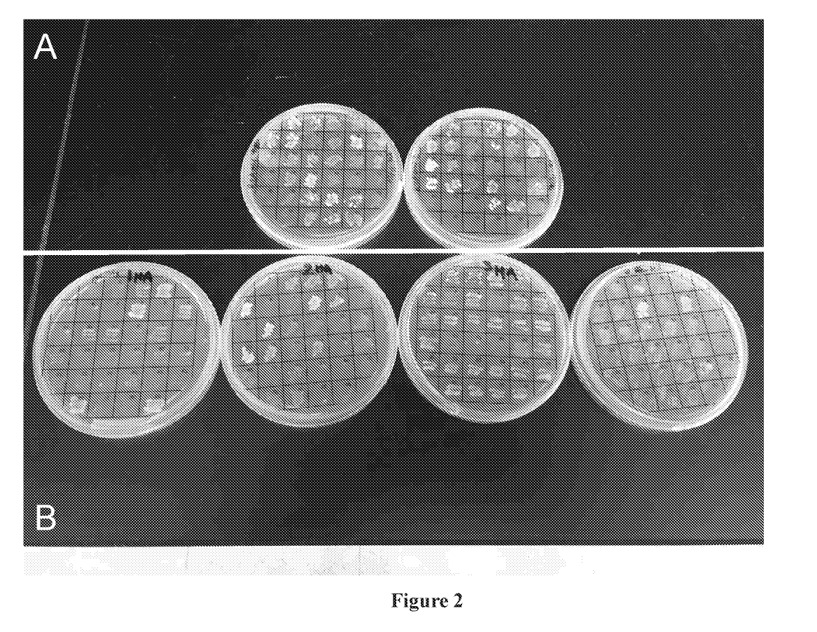

FIG. 2 shows example results from this study. Panel A of FIG. 2 illustrates the control arm of the study. Panel A (above the white line) shows two R2A plates (not containing humic acid), onto which individual colonies that had formed after plating soil on R2A plates (without humic acid), were transferred. The colonies were transferred from the original R2A plates to the R2A plates shown in panel A using sterile toothpicks. A different individual colony from the original R2A plates was transferred to each of the 32 outlined squares on each of the two R2A plates shown in FIG. 2A. The data in FIG. 2A show that approximately 57 of the 64 colonies (about 89%) transferred from the original R2A plates did subsequently grow on the R2A plates to which the original colonies were transferred.

Panel B of FIG. 2 illustrates the experimental arm of the study. Panel B (below the white line) shows four R2A plates (not containing humic acid) onto which individual colonies, that had formed after plating soil on R2A plates that contained humic acid, were transferred. The colonies were transferred from the original R2A plates containing humic acid to the R2A plates shown in panel B using sterile toothpicks. A different individual colony from the original R2A plates containing humic acid was transferred to each of the 32 outlined squares on each of the four R2A plates shown in FIG. 2B. The data in FIG. 2B show that approximately 114 of the 128 colonies (again about 89%) transferred from the original R2A plates containing 0.5% humic acid did subsequently grow on the R2A plates to which the original colonies were transferred.

These results indicate that the efficiency of growing microbes, originally isolated on media containing humic acid, on media that does not contain humic acid, is relatively high (almost 90%). This efficiency is about the same as that of growing bacteria, originally isolated on media not containing humic acid, subsequently on media that does not contain humic acid.

We have observed that some of the bacteria originally isolated on media containing humic acid (approximately 10%), appear to subsequently grow very slowly once the humic acid is removed. We have also observed an occasional bacterial colony, originally isolated on media containing humic acid, that does not appear to grow once the humic acid is removed.

Generally, therefore, we observe that humic acid can be removed from microbial media, and that microbes originally isolated on medium containing humic acid will still proliferate.

### Example 7. Increased Diversity of Bacteria Isolated from Soil Using Media Containing Humic Acid

The foregoing studies demonstrated that humic acid increased the efficiency with which microbes from environmental samples formed colonies and could be isolated from the samples. In Examples 7 and 8 herein, studies are described that were designed to determine whether humic acids increased the diversity of microbes isolated from environmental samples, as compared to microbial diversity when humic acid was not used in the isolation.

To perform these studies, microbial colonies were obtained after culturing soil sample B (described in Example 2) on either R2A plates, or R2A plates that contained 0.5% humic acid (weight/volume; Sigma-Aldrich No. 53680), as described in Example 3. Cultures of each isolated clone were sent to GENEWIZ, Inc. (Research Triangle Park, N.C., USA), where 16S rRNA gene sequencing was performed (http://www.genewiz.com/public/16S_rRNA.aspx). Alternatively, cultures were sent to MIDI Labs, Inc. (Newark, Del., USA) for 16S rRNA sequencing (http://www.midilabs.com/dna-sequencing).

The goal for our analysis (analysis is described below, and data from the analysis is shown in Table 9) was to analyze an equivalent number of 16S rRNA sequences from microbes isolated on R2A plates as sequences from microbes isolated on R2A plates that contained humic acid, to determine identities of the isolated microbes. However, as we sent microbes out for 16S rRNA sequencing, we received sequence data back from the vendors in groups that contained different numbers of acceptable reads, depending on the quality of the sequencing. A sequence was deemed acceptable and included in our analysis if the read from the 16S rRNA contained at least 1000 consecutive nucleotides. Once a group of sequences was received from a vendor, we included all acceptable sequences within the group in our analysis. Our analysis (Table 9) therefore, included the first 48 acceptable 16S rRNA sequences from microbes isolated using R2A plates, and the first 55 acceptable 16S rRNA sequences from microbes isolated using R2A plates that contained humic acid.

For our analysis, acceptable 16S rRNA sequences from the isolated microbes (i.e., “query sequences”) were used to query the NCBI BLASTN database (https://blast.ncbi.nlm.nih.gov/Blast.cgi?PAGE_TYPE=BlastSearch) for sequences included in the database (herein called “retrieved sequences”) that were most identical to the query sequence. When a retrieved sequence possessed at least 96% identity to the query sequence, over at least 99% of the query sequence length, the microbe from which the query sequence was obtained was assigned to the taxonomic genus of the microbe from which the highest identity retrieved sequence originated. In our analysis, the exception to this rule was that, if the database record for highest identity retrieved sequence indicated that the retrieved sequence came from an unculturable microbe, then the isolated microbe from which the query sequence was obtained was classified as “unculturable,” rather than as a member of a particular genus.

Additionally, as part of our analysis, when a query sequence did not return a retrieved sequence with at least 96% identity to the query sequence, over at least 99% of the query sequence length, the microbe from which the query sequence was obtained (i.e., one of our isolated microbes) was considered to be a microbe that was previously unknown. In other words, in this instance, we concluded not only that we had identified a microbe that no one had previously identified, but that we had successfully cultured this microbe using media containing humic acid.

Table 9 shows the results of the analysis. These data include 48 microbes isolated from R2A plates and 55 microbes isolated from R2A plates containing humic acid.

TABLE 9
Genera represented by microbes isolated
from R2A or R2A + humic acid (HA) media

| | Number of microbes | Number of microbes |
|---|---|---|
| | isolated from | isolated from |
| Genus | R2A¹ | R2A + HA² |
| Arthrobacter | 2 | 0 |
| Bacillus | 4 | 0 |
| Burkholderia | 3 | 6 |
| Caenimonas | 0 | 1 |
| Dermacoccus | 0 | 1 |
| Duganela | 1 | 0 |
| Janthinobacteria | 1 | 0 |
| Leifsonia | 1 | 0 |
| Lysinibacillus | 0 | 1 |
| Massilia | 26 | 7 |
| Methylobacterium | 0 | 1 |
| Mucilaginibacter | 1 | 2 |
| Nocardioides | 0 | 2 |
| Novosphingobium | 0 | 1 |
| Paenibacillus | 0 | 1 |
| Phycicoccus | 0 | 4 |
| Ramlibacter | 1 | 5 |
| Rhizobacter | 0 | 1 |
| Rugamonas | 0 | 1 |
| Sphingomonas | 1 | 4 |
| Streptococcus | 1 | 0 |
| Streptomyces | 2 | 2 |
| Terrabacter | 3 | 4 |
| Tetrasphaera | 0 | 4 |
| Unculturable³ | 0 | 4 |
| Unibacterium | 1 | 0 |
| Unknown⁴ | 0 | 3 |
| Total number of microbes | 48 | 55 |

¹Total isolates from R2A plates were 48.
²Total isolates from R2A + HA plates were 55.
³Unculturable means that retrieved sequences with the highest identities (at least 96%) to the query sequence were identified as “unculturable” in the BLASTN database.
⁴Unknown means that no retrieved sequence having at least 96% identity to the query sequence was found.

As indicated in Table 9, 48 microbes were isolated from R2A plates and included in the analysis. Among the 48 microbes, 14 different genera were represented. One indicator of the diversity of genera obtained from R2A plates in this study is an estimate of the average number of microbes that have to be isolated before an additional genus is identified. This can be determined by dividing the number of total isolated microbes (i.e., 48) by the total number of genera identified in the study (i.e., 14). From this study, 3.4 microbes were isolated before an additional genus was identified. Seven of the 14 different genera isolated from R2A plates were not represented among the 55 microbes isolated from R2A+HA plates. Additionally, among the 48 microbes isolated from R2A plates, no microbes were classed as unculturable and no microbes were previously unknown.

Also as indicated in Table 9, 55 microbes were isolated from R2A+HA plates and included in the analysis. Among the 55 microbes, 18 different genera were represented. Dividing the number of total isolated microbes (i.e., 55) by the total number of genera identified in the study (i.e., 18), it was determined that 3.1 microbes were isolated before an additional genus was isolated. Because fewer microbes were isolated per genus from R2A+HA plates (i.e., 3.1) than from R2 plates (i.e., 3.4), the diversity of microbes isolated from media containing humic acid is estimated to be higher than that isolated from media without humic acid. Additionally, 11 of the 19 different genera isolated from R2A+HA plates were not represented among the 48 microbes isolated from R2A plates.

Of significance is that, among the 55 microbes isolated using media containing humic acid, 4 of the microbes (4/55≈7%) were previously known as unculturable. This disclosure is the first report of these 4 organisms being cultured. In contrast, no unculturable organisms (0/48) were isolated in this study in the absence of humic acid.

Additionally, 3 out of the 55 microbes (over 5%) isolated using humic acid were previously unknown. This disclosure is the first report of the existence of these microbes. In contrast, no unknown microbes (0/48) were isolated in this study in the absence of humic acid.

Looking at the data in another way, almost 13% (7/55) of the microbes isolated using humic acid were either never before reported to have been cultured or were previously not known. Not one unculturable or previously unknown organism (0/48) was isolated when humic acid was not used. Note that as more organisms are analyzed within a group (55 for humic acid vs. 48 for no humic acid), the higher the probability that infrequent events (unculturable and previously unknown organisms) will be detected. However, the increased number of organisms in the humic acid group (55 vs. 48 in the non-humic acid group) does not account for the difference in the number of unculturable/previously unknown microbes between the humic acid group (7) and the non-humic acid group (0) in this study.

In total, these results support the conclusion that inclusion of humic acid in the growth media increases the diversity of isolated microbes as compared to the absence of humic acid.

### Example 8. Unculturable and Unknown Microbes

After the studies described in Example 7 were completed, we continued to isolate additional microbes from soil sample B using R2A plates that contained 0.5% humic acid. Seventy additional microbe isolates were obtained using humic acid. Nucleotide sequences for 16S rRNA was obtained from the 70 additional microbes. In this Example 8, we report on the unculturable and previously unknown microbes discovered in the 125 total microbes (55 described in Example 7 and 70 additional described in Example 8). These studies were carried out using the procedures already described.

Table 10 includes some information on these microbes. Column 1 of the table indicates whether an isolated microbe has been determined to be unculturable or previously unknown. These determinations were made using the analysis described in Example 7. Column 2 of Table 10 shows the numerical indicator for each microbe. Column 3 indicates the number of consecutive nucleotides present in the 16 rRNA sequence from the isolated microbes (i.e., the sequence used to query the database). Column 4 is the accession number of the retrieved sequence with the highest identity to the 16S rRNA sequence obtained from the isolated microbe. Column 5 includes a general description, obtained from the database record of the retrieved sequence, of the microbe from which the retrieved sequence originated.

TABLE 10
Unculturable and previously unidentified microbes
isolated using humic acid, and their closest 16S
rRNA matches obtained from BLASTN queries

| | | | Accession | |
|---|---|---|---|---|
| | | | number of re- | Description of |
| | Organ- | 16S | trieved sequence | microbe from |
| | ism | rRNA | with highest | which retrieved |
| Organism | desig- | contig | identity to | sequence |
| type | nation | length | query sequence | originated |
| Unculturable | 180 | 1239 | KM456152 | Unculturable |
| | | | | bacterium from pig |
| | | | | deep litter system |
| Unculturable | 209 | 1246 | AB696166 | Unculturable |
| | | | | bacterium from |
| | | | | environmental |
| | | | | sample |
| Unculturable | 234 | 1267 | EF516144 | Uncultured |
| | | | | bacterium from |
| | | | | soil system |
| Unculturable | 235 | 1214 | AB608698 | Uncultured |
| | | | | bacterium from rice |
| | | | | paddy sample |
| Unculturable | 255 | 1444 | KC683249 | Uncultured |
| | | | | bacterium from |
| | | | | river sample |
| Unculturable | 260 | 1270 | JF198713 | Uncultured |
| | | | | bacterium from |
| | | | | environmental |
| | | | | sample |
| Unculturable | 267 | 1237 | JF214882 | Uncultured |
| | | | | bacterium from |
| | | | | environmental |
| | | | | sample |
| Unculturable | 275 | 1277 | JF198713 | Uncultured |
| | | | | bacterium from |
| | | | | human skin sample |
| Previously | 223 | 1446 | JF833841 | Unculturable alpha |
| unknown | | | | proteobacterium |
| Previously | 257 | 1446 | DQ984596 | Unculturable |
| unknown | | | | bacterium from |
| | | | | environmental |
| | | | | sample |
| Previously | 281 | 1229 | LN571244 | Unculturable |
| unknown | | | | bacterium from leaf |
| | | | | cutter sample |

The data in Table 10 indicate that, of 125 organisms isolated from soil using humic acid, 8 of the strains (over 6%) were never before reported to be cultured and 3 of the strains (over 2%) have never before been described. Therefore, almost 9% of the 125 organisms isolated using humic acid were either previously thought to be unculturable or are new.

Additionally, bacteria from at least 28 known genera were among the 125 organisms. These genera included: Actinotalea, Amycolatopsis, Aquabacterium, Bacillus, Burkholderia, Caenimonas, Dermacoccus, Leifisonia, Lysinibacillus, Marmoricola, Massilia, Methylobacterium, Mucilaginibacter, Nocardia, Nocardioides, Novosphingobium, Paenibacillus, Phycicoccus, Ramlibacter, Rhizobacter, Rugamonas, Sphingomonas, Streptomyces, Terrabacter, Tetrasphaera, Tumebacillus, and Variovorax.

### Example 9. Fractionation of Humic Acid and Activity

Humic acid (7 g of Sigma-Aldrich No. 53680) was mixed with 150 ml of 0.1 M ammonium bicarbonate and pH adjusted to 9 using ammonium hydroxide. About 105 ml of the mixture was centrifuged at 7,000×g for 10 minutes in a preparative centrifuge. The supernatant was centrifuged through a 5,000 molecular weight cut-off filter (Corning® Spin-X® UF 20 ml Centrifugal Concentrator, 5,000 MWCO Membrane) at 5,000×g at 20° C. for 30 minutes. The flow-through was centrifuged through the filter 3 additional times. The material retained on the filter was suspended in buffer and pH adjusted to 7 using 10% acetic acid. The material was lyophilized. Subsequently, the activity of humic acid that resulted in the increased efficiency of plating of microbes from soil samples was shown to be present in this sample that was retained on the 5,000 molecular weight cut-off filter. We conclude that the majority of the activity in humic acid that increases efficiency of plating of microbes from soil samples is 5,000 molecular weight or above.

While example compositions, methods, and so on have been illustrated by description, and while the descriptions are in considerable detail, it is not the intention of the applicants to restrict or in any way limit the scope of the application. It is, of course, not possible to describe every conceivable combination of components or methodologies for purposes of describing the compositions, methods, and so on described herein. Additional advantages and modifications will readily appear to those skilled in the art. Therefore, the invention is not limited to the specific details and illustrative examples shown and described. Thus, this application is intended to embrace alterations, modifications, and variations that fall within the scope of the application. Furthermore, the preceding description is not meant to limit the scope of the invention.

To the extent that the term “includes” or “including” is employed in the detailed description or the claims, it is intended to be inclusive in a manner similar to the term “comprising” as that term is interpreted when employed as a transitional word in a claim. Furthermore, to the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

### Example Embodiments of the Invention

1. A method, comprising, consisting essentially of, or consisting of:

isolating an unculturable microbe or unknown microbe from an environmental sample using a medium that includes humic acid, a salt thereof, an analog thereof, or peat.

2. The method of embodiment 1, where the unculturable microbe or unknown microbe, after it has been isolated, is capable of being cultured using a medium that does not include humic acid, a salt thereof, an analog thereof, or peat.
3. The method of any one of embodiments 1 and 2, including, after the isolating step:

culturing the unculturable microbe or the unknown microbe using a medium that does not include humic acid, a salt thereof, an analog thereof, or peat.

4. The method of any one of embodiments 1-3, where the medium used for the isolating step includes the humic acid.
5. The method of any one of embodiments 1-4, where a concentration of the humic acid in the medium used for the isolating step is greater than 0% and less than about 5% (weight/volume).
6. The method of any one of embodiments 1-5, where the humic acid includes Sigma-Aldrich No. 53680.
7. The method of any one of embodiments 1-3, where the medium used for the isolating step includes the salt of humic acid.
8. The method of any one of embodiments 1-3, and 7, where a concentration of the salt of humic acid in the medium used for the isolating step is greater than 0% and less than about 0.25% (weight/volume).
9. The method of any one of embodiments 1-3, 7, and 8, where the salt of humic acid includes Sigma-Aldrich No. H16752.
10. The method of any one of embodiments 1-9, where the medium used for the isolating step includes a gelling agent.
11. The method of embodiment 10, where the gelling agent includes agar.
12. The method of any one of embodiments 1-11, where isolating includes formation of a colony of the unculturable microbe or unknown microbe on the medium after about 2-3 days incubation at about 30° C. in an ambient atmosphere, the medium including agar.
13. The method of any one of embodiments 1-12, where the environmental sample includes soil or water.
14. The method of any one of embodiments 1-13, where the medium used for the isolating step includes ISP2, Jensen's, LB, NA, R2A, or TSA.
15. The method of any one of embodiments 1-14, including, prior to the isolating step:

selecting a medium for isolating a microbe, recognizing that efficiency or diversity of microbes cultured from an environmental sample may be increased by using medium containing humic acid, a salt thereof, or an analog thereof.

16. The method of any one of embodiments 1-15, where the unculturable microbe or unknown microbe excludes microbes from the order Actinomycetales.
17. The method of any one of embodiments 1-16, where the unculturable microbe or unknown microbe excludes microbes from the phyla Acidobacteria and Verrucomicrobia. 18. The method of embodiments 17, where the Acidobacteria belongs to subdivision 1 and where the Verrrucomicrobia belongs to subdivision 4.
19. The method of any one of embodiments 1-18, where the unculturable microbe and the unknown microbe are prokaryotes.
20. The method of any one of embodiments 1-19, where the unculturable microbe and the unknown microbe are bacteria or archaea.
21. A method, comprising, consisting essentially of, or consisting of:

culturing a bacterium from an environmental sample on or in a medium containing humic acid, a salt thereof, or an analog thereof; and

subsequently culturing the bacterium on or in a medium that does not contain humic acid, a salt thereof, or an analog thereof.

22. The method of embodiment 21, where the culturing from the environmental sample on or in the medium containing humic acid, a salt thereof, or an analog thereof, occurs in absence of prior enrichment procedures or prior culturing on or in a medium not containing humic acid, a salt thereof, or an analog thereof.
23. The method of any one of embodiments 21 and 22, where the bacterium is an unculturable or an unknown bacterium.
24. A method for isolating a bacterium from an environmental sample, comprising, consisting essentially of, or consisting of:

plating a portion of the environmental sample on an medium containing a gelling agent that contains humic acid, a salt thereof, or an analog thereof such that bacterial colonies form on the medium;

where the bacterial colonies exclude the order Actinomycetales and the phyla Acidobacteria or Verrucomicrobia.

25. The method of embodiment 24, where bacteria from the colonies are unculturable bacteria or unknown bacteria.
26. The method of any one of embodiments 24 and 25, where bacteria from the colonies are capable of being cultured on a medium that does not contain humic acid, a salt thereof, or an analog thereof.
27. The method of any one of embodiments 24-26, including, subsequent to the plating step:

culturing bacteria from the colonies using a medium that does not contain humic acid, a salt thereof, or an analog thereof.

28. The method of any one of embodiments 24-27, including:

prior to the plating step, recognizing that a probability of isolating an unculturable bacterium or unknown bacterium may be increased by using a medium containing humic acid, a salt thereof, or an analog thereof.

29. The method of any one of embodiments 24-28, including:

obtaining at least a partial sequence of a 16S rRNA from the bacteria from the colonies.

30. The method of any one of embodiments 24-29, where the bacteria from the bacterial colonies are members of the genus Actinotalea, Amycolatopsis, Aquabacterium, Bacillus, Burkholderia, Caenimonas, Dermacoccus, Leifisonia, Lysinibacillus, Marmoricola, Massilia, Methylobacterium, Mucilaginibacter, Nocardia, Nocardioides, Novosphingobium, Paenibacillus, Phycicoccus, Ramlibacter, Rhizobacter, Rugamonas, Sphingomonas, Streptomyces, Terrabacter, Tetrasphaera, Tumebacillus, and Variovorax.
31. A method for culturing bacteria from an environmental sample, comprising, consisting essentially of, or consisting of:

plating the environmental sample, or dilution thereof, on an agar-containing medium containing humic acid, a salt thereof, or an analog thereof, such that bacterial colonies form on the medium, the bacteria in the environmental sample not having been subjected to prior enrichment or to prior growth; and

transferring one or more of the bacterial colonies to a medium not containing humic acid, a salt thereof, or an analog thereof, such that the bacterial colonies grow on or in the medium not containing humic acid, a salt thereof, or analogs thereof.

32. The method of embodiment 30, including:

obtaining at least part of a 16S rRNA sequence from one of the bacterial colonies; and

determining a taxonomic grouping of the bacterial colony based, at least in part, on the 16S rRNA sequence.

33. The method of embodiment 32, where an inability to determine a taxonomic grouping indicates the bacterium is unculturable or was previously unknown.
34. The method of any one of embodiments 31-33, where the bacterial colony is not from the order Actinomycetales and is not from the phyla Acidobacteria or Verrucomicrobia.
35. The method of any one of embodiments 31-34, where, prior to the plating step, recognizing that medium containing humic acid, a salt thereof, or an analog thereof, could facilitate culturing bacteria from an environmental sample.
36. A method for isolating bacteria from a soil environmental sample, comprising, consisting essentially of, or consisting of:

recognizing that addition of humic acid, a salt thereof, or an analog thereof to a bacterial medium could increase the efficiency and/or diversity of the bacteria isolated from the soil environmental sample;

culturing bacteria from a portion of the soil environmental sample on an agar-containing bacterial medium containing humic acid, a salt thereof, or an analog thereof, such that bacterial colonies form on the bacterial medium; and

subsequently culturing bacteria from the bacterial colonies on or in a medium that does not contain humic acid, a salt thereof, or an analog thereof.

37. The method of embodiment 36, including:

obtaining at least a partial nucleotide sequence of a 16S rRNA from one of the bacterial colonies; and

determining, at least in part based on the 16S rRNA sequence, that bacteria from one of the bacterial colonies was previously unknown or is unculturable.

38. The method of any one of embodiments 36 and 37, where the isolated bacteria are not from the order Actinomycetales.
39. The method of any one of embodiments 36-38, where the isolated bacteria are not from the phyla Acidobacteria or Verrucomicrobia.
40. The method of any one of embodiments 36-39, where the humic acid includes Sigma-Aldrich No. 53680 and the salt of humic acid includes Sigma-Aldrich No. H16752.
41. A microbe or bacterium isolated by the method of any one of embodiments 1-40.
42. A petri dish comprising a medium capable of supporting growth of a microbe, the medium containing humic acid, a salt thereof, or an analog thereof, and a gelling agent, and including a colony of an unculturable or unknown microbe that has formed on the medium.

1. A method, comprising:
isolating an unculturable microbe or unknown microbe from an environmental sample using a medium that includes humic acid, a salt thereof, an analog thereof, or peat.

2. (canceled)

3. The method of claim 1, including, after the isolating step:
culturing the unculturable microbe or the unknown microbe using a medium that does not include humic acid, a salt thereof, an analog thereof, or peat.

4. (canceled)

5. The method of claim 1, where a concentration of the humic acid in the medium used for the isolating step is greater than 0% and less than about 5% (weight/volume).

6. (canceled)

7. The method of claim 1, where the medium used for the isolating step includes the salt of humic acid.

8-9. (canceled)

10. The method of claim 1, where the medium used for the isolating step includes a gelling agent.

11. (canceled)

12. The method of claim 1, where isolating includes formation of a colony of the unculturable microbe or unknown microbe on the medium after about 2-3 days incubation at about 30° C. in an ambient atmosphere, the medium including agar.

13. The method of claim 1, where the environmental sample includes soil or water.

14-18. (canceled)

19. The method of claim 1, where the unculturable microbe and the unknown microbe are prokaryotes.

20. (canceled)

21. A method, comprising:
culturing a bacterium from an environmental sample on or in a medium containing humic acid, a salt thereof, or an analog thereof; and
subsequently culturing the bacterium on or in a medium that does not contain humic acid, a salt thereof, or an analog thereof.

22. The method of claim 21, where the culturing from the environmental sample on or in the medium containing humic acid, a salt thereof, or an analog thereof, occurs in absence of prior enrichment procedures or prior culturing on or in a medium not containing humic acid, a salt thereof, or an analog thereof.

23. The method of claim 21, where the bacterium is an unculturable or an unknown bacterium.

24-30. (canceled)

31. A method for culturing bacteria from an environmental sample, comprising:
plating the environmental sample, or dilution thereof, on an agar-containing medium containing humic acid, a salt thereof, or an analog thereof, such that bacterial colonies form on the medium, the bacteria in the environmental sample not having been subjected to prior enrichment or to prior growth; and
transferring one or more of the bacterial colonies to a medium not containing humic acid, a salt thereof, or an analog thereof, such that the bacterial colonies grow on or in the medium not containing humic acid, a salt thereof, or analogs thereof.

32. The method of claim 30, including:
obtaining at least part of a 16S rRNA sequence from one of the bacterial colonies; and
determining a taxonomic grouping of the bacterial colony based, at least in part, on the 16S rRNA sequence.

33. The method of claim 32, where an inability to determine a taxonomic grouping indicates the bacterium is unculturable or was previously unknown.

34-42. (canceled)
